(19) United States
(12) Patent Application Publication
(10) Pub. No.: US 2004/0215315 A1
(43) Pub. Date: Oct. 28, 2004

(54) Drug-eluting stent with sheath and balloon deployment assembly

(76) Inventors: Ryan A. Jones, Santa Rosa, CA (US); John D. Kantor, Santa Rosa, CA (US)

Correspondence Address: FRANK C. NICHOLAS, CARDINAL LAW GROUP, Suite 2000, 1603 Orrington Avenue, Evanston, IL, 60201, US

(21) Appl. No.: 10/831,081

(22) Filed: Apr. 23, 2004

Related U.S. Application Data
(60) Provisional application No. 60/465,396, filed on Apr. 25, 2003.

(51) Int. Cl. A61F 2/06

(52) U.S. Cl. 623/1.11; 623/1.42; 623/1.46

(57) ABSTRACT

The present invention provides a system for treating a vascular condition, including a catheter and a stent deployment assembly coupled to the catheter. The stent deployment assembly includes a coated stent with a stent framework and a drug-polymer coating on at least a portion of the stent framework. An expandable sheath is positioned adjacent to an inner surface of the coated stent, and at least one balloon is positioned within the expandable sheath. The expandable sheath expands against the stent framework as the balloon is inflated to deploy the coated stent within a vessel.

### RELATED APPLICATION

This application claims priority to U.S. Provisional Application No. 60/465,396, “Drug-eluting Stent with Sheath and Balloon Deployment Assembly” to Ryan A. Jones and John D. Kantor, filed Apr. 25, 2003, the entirety of which is incorporated by reference.

### FIELD OF THE INVENTION

This invention relates generally to catheter deployment of drug-coated stents. More specifically, the invention relates to a stent deployment assembly including an expandable, elastic sheath and one or more balloons.

### BACKGROUND OF THE INVENTION

An increasing number of stents for treating vascular conditions are being coated with pharmaceutical drugs and protective materials for controlled time-delivery of the therapeutic agents. Medical research indicates a greater effectiveness of vascular stents when the stents are coated with pharmaceutical drugs that help prevent or treat medical conditions. These drugs may be released from a coating while in the body, delivering their patent effects at the site where they are most needed. The drugs may be mixed, for example, within drug-polymers or encased by polymeric coatings on the stents. Stent coatings with various families of drug polymer chemistries have been used to increase the effectiveness of stenting procedures and to control drug-elution properties.

Unfortunately, the use of current catheter balloons with the drug-coated stents may lead to several problems. The unfolding of a balloon during the deployment of a stent can cause excessive shear forces on the inner surface of the stent. Such a shear may cause the tearing or abrasion of the stent coating, which may include, for example, drugs mixed with polymers.

Controlling the amount of inflation and deflation of catheter balloons is critical to the proper exertion of an expandable stent against a body vessel. One design for controlling a balloon is described by Hillstead in “Dilatation Balloon within an Elastic Sleeve”, U.S. Pat. No. 5,366,472, issued Nov. 22, 1994. A double-walled dilation balloon has an inner inflatable balloon wall and an outer balloon wall with a space in between. In one embodiment of the balloon catheter assembly, an outer elastic sleeve is positioned about and around the balloon, exerting a compressive force against the balloon.

Coatings and lubricants have been used in some balloon and catheter configurations to help with the placement and movement of a medical device such as a catheter balloon. One such coating composition that is used on various articles including medical devices is described by Opolski in “Articles Prepared from Water-Based Hydrophilic Coating Compositions”, U.S. Pat. No. 6,238,799, issued May 29, 2001.

Some balloon configurations have used varying textures of balloon material or lubricants to control the movement of the balloon. The friction level on the exterior surface of a catheter balloon can be different between the collapsed or pre-deployed state and an inflated balloon state. Abele et al. in “Balloon Catheter”, U.S. Pat. No. 6,010,480, issued Jan. 4, 2000 and “Balloon Catheter”, U.S. Pat. No. 5,693,014, issued Dec. 2, 1997, describe two such configurations. The balloon catheter has a first exterior surface with a given coefficient of friction and a second exterior surface with a greater coefficient of friction. The first exterior surface provides a certain level of friction during transfer of the collapsed balloon and then when the balloon is inflated, the second surface dominates the first surface and produces a different level of friction.

Over the last decade, various configurations of catheter balloon assemblies have been suggested, using balloons of multiple textured materials, lengthwise pleats or folds, or accordion-like lobes. A balloon with smooth longitudinal strips where folds are created is described in “Catheter Balloon Having Raised Radial Segments”, Ravenscroft, et al., U.S. Pat. No. 6,110,192, issued Aug. 29, 2000. A textured dilation balloon with a reduced texture longitudinal strip causes preferential controlled folding along the strip to create curved wings that fold together upon further deflation. The curved, folded wings result in a decreased profile having wing edges oriented so as to decrease contact with conduit walls during retraction of the catheter balloon. A preferred embodiment has raised radial ridges extending around a portion of the circumference of the balloon and a smooth longitudinal surface radially there between.

A catheter with multiple pleats that are folded or wrapped circumferentially around the catheter is described in “Six-Pleated Catheter Balloon and Device for Forming Same”, Butaric, et al., U.S. Pat. No. 6,033,380 issued Mar. 7, 2000. The balloon pleats extend about one-quarter of the circumference of the catheter shaft.

A balloon with a generally cylindrical shape and four ribs longitudinally and equidistantly arranged around an axis is disclosed in “Medical Balloon Folding into Predetermined Shapes and Method”, Campbell, et al., U.S. Pat. No. 5,478,319 issued Dec. 26, 1995. When fully inflated, the balloon is a continuous body of balloon material with ribs formed of stressed balloon material and webs of less stressed balloon material between the ribs. The balloon is expanded from a folded condition for insertion into the body part to an expanded condition with a generally cylindrical shape and a diameter substantially greater than the folded condition.

A tri-fold catheter balloon is disclosed by Tsukashima et al. in “Tri-fold Balloon for Dilatation Catheter and Related Method”, U.S. Pat. No. 5,350,361, issued Sep. 27, 1994. The balloon has softened longitudinal creases defined by three flaps, which help a symmetrical deflation of the balloon. Three pins in a pyramid-type stack formation and a longitudinal heating element within an interstitial channel help symmetrical inflation and deflation of the balloon.

A catheter balloon with three or more folded wings or flaps is disclosed by Euteneuer et al. in “Dilatation Catheter with Tri-Fold Balloon”, U.S. Pat. No. 5,342,307, issued Aug. 30, 1994. The wings of the balloon are wrapped around circumferentially in a deflated state with a minimized outer diameter. Another catheter balloon with pleats and folds is described by Davey in “Pleated Balloon Dilatation Catheter and Method of Use”, U.S. Pat. No. 5,318,587, issued Jun. 7, 1994.

A lobed configuration of a catheter balloon is described in “Balloon Catheter with Lobed Balloon and Method for Manufacturing such a Catheter”, Weber, et al., U.S. Pat. No. 5,759,172, granted Jun. 2, 1998. Relatively stiff sections are alternated with pliable sections of the balloon, the pliable sections forming lobes in an expanded state of the balloon. A catheter assembly with a catheter, an accordion-like sheath, and a balloon inside is disclosed in “Catheter Assembly and Related Method”, Knoll, et al., U.S. Pat. No. 5,242,398, issued Sep. 7, 1993.

While the designs of the balloon deployment systems mentioned above provide for inflation and deflation of a catheter balloon, they may or may not be successfully implemented with the newer type of drug-coated stents, which create additional concerns of keeping the coatings intact. Therefore, it is desirable to have a stent deployment system and method whereby the drug coating of the stent is protected from the sheering forces of a catheter balloon or any other part of a catheter assembly, and the coating or coatings of the stent are delivered intact to the point of deployment without abrasion or loss of material.

### SUMMARY OF THE INVENTION

One aspect of the invention provides a system for treating a vascular condition including a catheter and a stent deployment assembly coupled to the catheter. The stent deployment assembly comprises a coated stent including a stent framework and a drug-polymer coating on at least a portion of the stent framework, an expandable sheath positioned adjacent to an inner surface of the coated stent, and one or more balloons positioned within the expandable sheath. The expandable sheath expands against the stent framework as the balloon is inflated to deploy the coated stent within a vessel.

Another aspect of the invention is a method of deploying a drug-polymer coated stent for a vessel, including the steps of inflating pleated portions of a balloon, expanding a sheath with the inflating pleated portions of the balloon, and deploying the drug-polymer coated stent with the expanding sheath.

Another aspect of the invention is a method of manufacturing a stent deployment assembly, including steps of positioning a balloon within an expandable sheath, the balloon including a plurality of pleats; placing the expandable sheath and the balloon inside a drug-polymer coated stent; and compressing the stent deployment assembly, wherein the drug-polymer coated stent is pressed against an outside surface of the expandable sheath.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

FIG. 1 is an illustration of a system for treating a vascular condition, in accordance with one embodiment of the current invention;

FIG. 2 is a cross-sectional view of a stent deployment assembly, in accordance with one embodiment of the current invention;

FIG. 3 is a cross-sectional view of a stent deployment assembly with a plurality of balloons, in accordance with one embodiment of the current invention;

FIG. 4 is a flow diagram of a method for deploying a drug-polymer coated stent in a vessel, in accordance with one embodiment of the current invention; and

FIG. 5 is a flow diagram of a method for manufacturing a stent deployment assembly, in accordance with one embodiment of the current invention.

### DETAILED DESCRIPTION OF THE PRESENTLY PREFERRED EMBODIMENTS

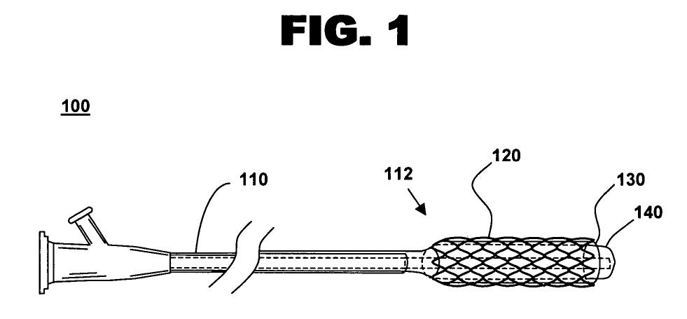

FIG. 1 illustrates a system for treating a vascular condition, in accordance with one embodiment of the present invention at 100. Vascular condition treatment system 100 includes a catheter 110 and a stent deployment assembly 112 coupled to catheter 110. Stent deployment assembly 112 includes a coated stent 120, an expandable sheath 130, and at least one balloon 140.

One or more therapeutic agents are in contact with polymeric coatings on coated stent 120. Vascular condition treatment system 100 may help treat, for example, heart disease, various cardiovascular ailments, and other vascular conditions by using catheter-deployed endovascular stents that have tailored polymeric coatings for controlling the timed-release properties of interdispersed or encased therapeutic agents. Treatment of vascular conditions may include the prevention or correction of various ailments and deficiencies associated with the cardiovascular system, the cerebrovascular system, urinogenital systems, biliary conduits, abdominal passageways and other biological vessels within the body.

Prior to the deployment of coated stent 120, expandable sheath 130 is positioned adjacent to an inner surface of coated stent 120, with at least one balloon 140 positioned within expandable sheath 130. During deployment, balloon 140 inflates and presses outwardly against expandable sheath 130. Expandable sheath 130 expands against coated stent 120, enlarging coated stent 120. Coated stent 120 expands as balloon 140 inflates and deploys coated stent 120 within a vessel in a body.

To inflate balloon 140, a liquid such as a saline solution or a contrast fluid containing radiopaque material is injected into one or more lumens within catheter 110 and into the interior of balloon 140. The liquid is pressurized with a controller external to catheter 110, inflating balloon 140 and coated stent 120 until the desired diameter of coated stent 120 is obtained. The pressure applied to the liquid is reduced and balloon 140 collapses, in part due to the reduction of applied pressure and in part due to compression forces generated by expandable sheath 130. Coated stent 120 generally retains its enlarged shape, pressing outwards against the vessel wall and being secured in part by the tissue bed and vascular wall surrounding coated stent 120. After successful deployment and implantation of coated stent 120, catheter 110 with balloon 140 and expandable sheath 130 are removed from the body. Expandable sheath 130 helps the collapse and refolding of balloon 140 prior to their retraction into guide catheter 110.

One or more lumens may be included inside catheter 110 to fill multiple balloons 140 positioned within expandable sheath 130. An inner member is typically included inside catheter 110 for a guide wire used to position catheter 110 in the body.

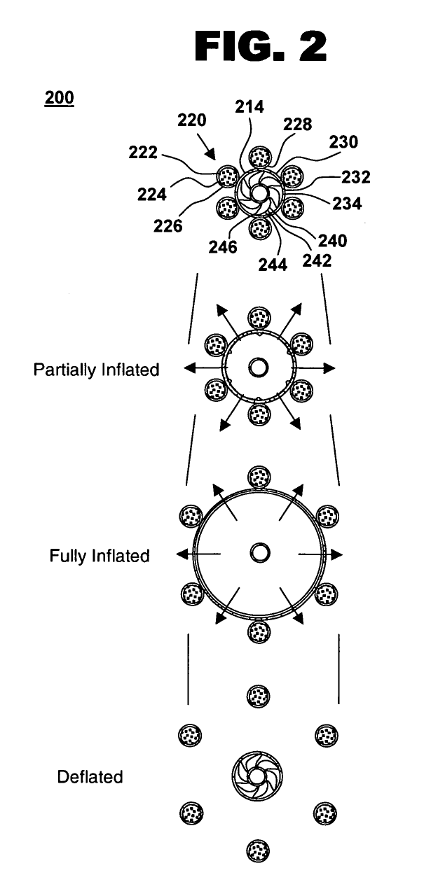

FIG. 2 shows a cross-sectional view of a stent deployment assembly, in accordance with one embodiment of the present invention at 200. Stent deployment assembly 200 includes a coated stent 220, an expandable sheath 230, and at least one balloon 240 positioned within expandable sheath 230. Expandable sheath 230 is positioned adjacent to an inner surface of coated stent 220. Shear forces on coated stent 220 due to an inflating balloon 240 are reduced by expandable sheath 230 that wraps and encompasses balloon 240. An inner member 214 may be centrally located within the stent deployment assembly to contain a guide wire that is inserted through stent deployment assembly 200 during the deployment of coated stent 220.

Coated stent 220 includes a stent framework 222 and a drug-polymer coating 224 on at least a portion of the stent framework. Stent framework 222 may comprise a polymeric base or a metallic base such as stainless steel, nitinol, tantalum, MP35N alloy, platinum, titanium, a suitable biocompatible alloy, a suitable biocompatible material, a suitable polymeric material, or a combination thereof. The polymeric base material may comprise any suitable polymer for biomedical stent applications, as is known in the art. Drug-polymer coating 224 may include or encapsulate one or more therapeutic agents.

Drug-polymer coating 224 may comprise one or more therapeutic agents dispersed within or encased by a polymeric coating, which are eluted from coated stent 220 with controlled time delivery after deployment of coated stent 220 within a body. A therapeutic agent is capable of producing a beneficial effect against one or more conditions including coronary restenosis, cardiovascular restenosis, angiographic restenosis, arteriosclerosis, hyperplasia, and other diseases and conditions. For example, the therapeutic agent can be selected to inhibit or prevent vascular restenosis, a condition corresponding to a narrowing or constriction of the diameter of the bodily lumen where the stent is placed. Drug-polymer coating 224 may comprise, for example, an antirestenotic drug, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, or combinations thereof. The elution rates of the therapeutic agents into the body and the tissue bed surrounding the stent framework are based on the constituency and thickness of drug-polymer coating 224, the nature and concentration of the therapeutic agents, the thickness and composition of cap coat 226, and other factors.

Expandable sheath 230 is positioned adjacent to an inner surface of coated stent 220. The materials for the elastic expandable sheath 230 are generally selected as to have a high degree of lubricity relative to the inner surface of coated stent 220. Expandable sheath 230 comprises an elastic material such as nylon, polyurethane, polyethylene terephthalate (PET), polyethylene, polytetrafluoroethylene (PTFE), expanded polytetrafluoroethylene (ePTFE), an elastane, a thermoplastic elastomer, a woven polymeric fabric, or an expandable polymeric sheet. Expandable sheath 230 may comprise, for example, a biodurable thermoplastic polyurethane elastomer such as ChronoFlex® developed by CT Biomaterials of Woburn, Mass., a segmented polyurethane such as Spandex made by DuPont, or an engineered polytetrafluoroethylene such as Gore-Tex made by W. L. Gore and Associates, Inc., of Newark, Del. Expandable sheath 230 extends along a central axis of coated stent 220, generally with a margin of additional material extending beyond each end of coated stent 220. Expandable sheath 230 is generally thin and cylindrical, wrapping around and encompassing balloon 240.

Balloon 240 comprises an elastic material such as polyurethane, polyethylene terephthalate (PET), or a thermoplastic elastomer, as is currently known in the art. Balloon 240 is generally cylindrical and elongated, and may include two or more folds or pleats 244. When balloon 240 is deflated, pleats 244 are collapsed and may be rolled atop one another to provide a small diameter cross-section. As balloon 240 is inflated, portions of pleats 244 are inflated and expand expandable sheath 230. Expandable sheath 230 expands against stent framework 222 as coated stent 220 is deployed.

Balloon 240 may include a plurality of pleats 244, molded, pressed, or otherwise formed into the balloon structure. In one embodiment, an apex 246 of each pleat 244 may be attached to an inner surface of expandable sheath 230. Pleat 244 may be attached to expandable sheath 230 at one or more points along the outermost portion of pleat 244. As balloon 240 is inflated, the attached portions of pleat 244 assist in uniform expansion of expandable sheath 230 and ensure that expandable sheath 230 remains coupled to the catheter when stent deployment assembly 200 is removed from the body after coated stent 220 is deployed. In another embodiment, a lubricious coating 242 is positioned on at least a portion of an outer surface of balloon 240. Balloon 240 is lubriciously or slidably coupled to expandable sheath 230, such that balloon 240 may slide freely against an inner surface of expandable sheath 230 when balloon 240 is inflated and subsequently deflated.

As illustrated, pleats 244 fill with fluid and enlarge as balloon 240 is inflated, pressing outwards against expandable sheath 230. Fluid ingresses into balloon 240 through an inflation lumen in the catheter and into balloon 240. As more pressure is applied to the fluid in balloon 240, pleats 244 may completely unfold so that balloon 240 contacts expandable sheath 230 uniformly. Further pressure may further enlarge balloon 240, expanding coated stent 220 to a larger diameter as coated stent 220 is deployed. As pressure is relieved, coated stent 220 separates from expandable sheath 230. Balloon 240 is deflated and pleats 244 reform and cause balloon 240 and expandable sheath 230 to fold back into the original, compact shape about inner member 214.

Smooth transitions between a compressed state, an expanded state where coated stent 220 is deployed, and back to a compressed state for subsequent retraction of balloon 240 and expandable sheath 230 from the vessel can be facilitated by inclusion of a lubricious coating. Potential damage to drug-polymer coating 224 is reduced with a hydrophilic coating on an inner surface of coated stent 220, expandable sheath 230, or balloon 240. The lubricious coating may be positioned at one or more interfaces between balloon 240, expandable sheath 230, and coated stent 220. For example, a lubricious coating 242 may be positioned on at least a portion of an outer surface of balloon 240, such as on one or both sides of pleats 244. In another example, a lubricious coating 232 may be positioned on an outer surface of expandable sheath 230. In another example, a lubricious coating 234 may be positioned on an inner surface of expandable sheath 230. A portion of the inner surface or outer surface of expandable sheath 230 may be coated to provide a desired amount of friction or contact force with coated stent 220 or balloon 240. In another example, lubricious coating 228 may be positioned on an inner surface of coated stent 220. Lubricious coating 228 may cover, for example, the inner surface of coated stent 220 in contact with expandable sheath 230. The lubricious coating may comprise a material such as phosphorylcholine, a hydrophilic coating, or a lubricious film. Other portions of coated stent 220 may also be coated with lubricious coating 228 to minimize the coefficient of friction between balloon 240 and coated stent 220. Patterns, stripes, and fractional coverage of the various surfaces with the lubricious coating may be used to control stent retention during delivery and deployment.

In another example, a polymeric cap coat 226 is disposed on top of drug-polymer coating 224 to provide additional protection from shear forces generated during stent deployment and to aid in the control of the elution rate of one or more therapeutic agents dispersed within or encased by the coatings. Cap coat 226 is selected to adhere well to the drug-polymer coating and to provide a controlled frictional interface between cap coat 226 and expandable sheath 230. Cap coat 226 may be any suitable polymeric cap coating material such as parylene, polyurethane, phenoxy, epoxy, polyimide, polysulfone, or pellathane.

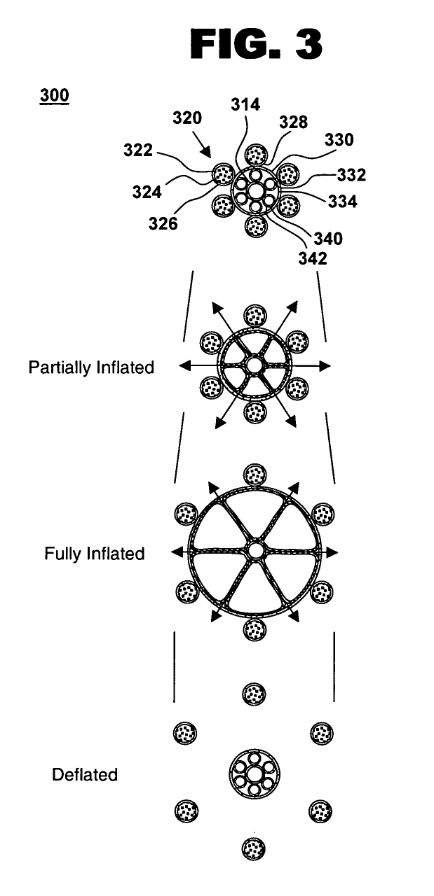

FIG. 3 shows a cross-sectional view of a stent deployment assembly with a plurality of balloons, in accordance with one embodiment of the present invention at 300. Stent deployment assembly 300 includes a coated stent 320, an expandable sheath 330 positioned adjacent to an inner surface of coated stent 320, and a plurality of balloons 340 positioned within expandable sheath 330. Expandable sheath 330 expands against coated stent 320 as one or more balloons 340 are inflated to deploy coated stent 320 within a vessel. Shear forces on coated stent 320 may be reduced by using multiple balloons, each having a single point of contact with the inner surface of coated stent 320 during deployment.

Balloons 340 are generally disposed around and attached to an inner member 314. Balloons 340 may be inflated and deflated with a pressurized fluid coupled to balloons 340 through a catheter, which is coupled to stent deployment assembly 300. As balloons 340 are inflated, they enlarge and press outwardly on expandable sheath 330. With applied pressure up to nine atmospheres and more, expandable sheath 330 expands against a stent framework 322 that may be coated with a drug-polymer coating 324. Stent framework 322 may be further coated with a cap coat 326 over drug-polymer coating 324, reducing the effect of shear stresses that may occur when coated stent 320 is deployed.

A lubricious coating such as phosphorylcholine, a hydrophilic coating, or a suitably lubricious film may be positioned to allow surfaces of balloons 340, expandable sheath 330, and coated stent 320 to slidably move against one another when coated stent 320 is expanded. For example, a lubricious coating 328 may be positioned on at least the inner surface of coated stent 320. A lubricious coating 332 may be positioned on an outer surface of expandable sheath 330. A lubricious coating 334 may be positioned on an inner surface of expandable sheath 330. A lubricious coating 342 may be positioned on at least a portion of an outer surface of one or more balloons 340.

As balloons 340 are inflated, expandable sheath 330 is expanded and presses outwardly on coated stent 320. When balloons 340 are fully inflated to the desired pressure, coated stent 320 deforms and develops a permanent set. As pressure applied to balloons 340 is diminished, expandable sheath 330 contracts and balloons 340 deflate. Coated stent 320 separates from expandable sheath 330 and balloons 340, and is retained at a directed location against a vascular wall while expandable sheath 330, balloons 340 and the catheter coupled to expandable sheath 330 and balloons 340 are retracted and removed from the body.

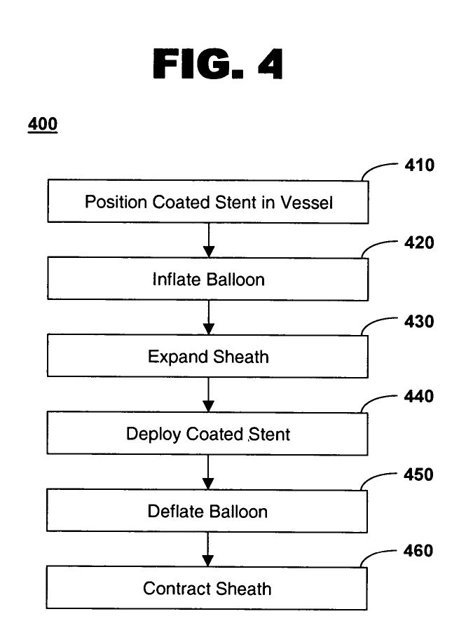

FIG. 4 shows a flow diagram of a method for deploying a drug-polymer coated stent in a vessel, in accordance with one embodiment of the present invention at 400. Coated stent deployment method 400 includes various steps to deploy a drug-polymer coated stent in a vessel in a body.

A drug-polymer coated stent is positioned in a vessel in the body, as seen at block 410. The vessel may be located in one of many vessels within the cardiovascular system, or in other vascular systems within the body such as the cerebrovascular system, the urinogenital system, biliary conduits, abdominal passageways, or peripheral vasculature. A catheter coupled to the drug-polymer coated stent in conjunction with a guide wire is inserted into one of the vessels of the body such as the femoral artery, and the coated stent is guided through one or more vessels into a directed location within the body. The coated stent position may be monitored, for example, using radiopaque markers or radiopaque fluid with associated x-ray imaging systems. The guide wire and catheter are manually manipulated through the vascular system to the desired location for stent deployment.

The balloons and any pleated portions are inflated, as seen at block 420. The balloons and pleated portions are filled with a liquid such as a contrast fluid that is fluidly coupled through the catheter from a source external to the body. As pressure is applied to the fluid, the balloons enlarge.

An expandable sheath is expanded, as seen at block 430. As the balloon enlarges, the expandable sheath is pressed outwardly with the inflating pleated portions of the balloon. As the balloon expands and is unfolded, the expandable elastic sheath minimizes the shear force on the inner diameter of the coated stent. The elastic material of the expanded sheath presses inwardly, keeping the sheath in contact with the at least one balloon. In one embodiment, pleated portions of the balloon are attached to the expandable sheath at one or more points, such as at the apex or outermost tip of each pleat. In another embodiment, the sheath is slidably or lubriciously coupled to the pleated portions of the balloon, the outer surface of the balloon and the inner surface of the sheath moving relatively easily against one another as the balloon is inflated and the sheath expands. The sheath is connected to the catheter along with the balloon to restrain sheath and balloon from aberrant movement. As the sheath expands, a coated stent surrounding the sheath expands.

The coated stent is deployed, as seen at block 440. The coated stent is deployed with the expanding sheath. The coated stent is enlarged and is secured against the tissue bed of the vascular wall. The size of the deployed stent is determined in part by the maximum pressure applied to the fluid when inflating the balloon.

The balloon and any pleated portions are deflated after the coated stent is deployed, as seen at block 450. The pressure applied to the interior of the balloon is reduced and the sheath contracts while deflating the balloon and pleated portions, the coated stent separating from the sheath and balloon.

The expandable sheath contracts, as seen at block 460. The contracting sheath squeezes out liquid from the balloon. Pleats on the balloon fold over one another to provide a compact cross section for removal. Liquid in the balloon may be pumped out, collapsing the balloon even further inside the expandable sheath. The expandable sheath, balloon and catheter are then withdrawn from the vessel.

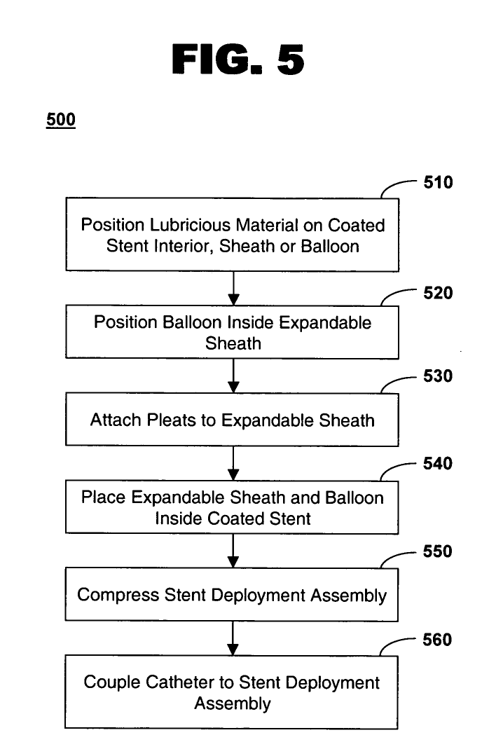

FIG. 5 shows a flow diagram of a method of manufacturing a stent deployment assembly, in accordance with one embodiment of the present invention at 500. Stent deployment assembly method 500 includes steps to manufacture a stent deployment assembly including a coated stent with a stent framework and a drug-polymer coating on at least a portion of the stent framework, an expandable sheath positioned adjacent to an inner surface of the coated stent, and one or more balloons positioned within the expandable sheath.

A lubricious material may be positioned on a balloon, a sheath, or a coated stent, as seen at block 510. The lubricious material helps to protect the integrity and improve the durability of stent coatings during delivery and deployment. The lubricious material may be positioned, for example, by coating at least a portion of the outer surface of the expandable sheath. The lubricious material may comprise, for example, phosphorylcholine, a hydrophilic coating, or a lubricious film. The lubricious material may be applied, for example, using any suitable application technique such as dipping, spraying, painting or brushing. The lubricious material may be dissolved or suspended in a suitable solvent such as isopropyl alcohol, ethanol, or methanol before application, applied, and then dried. The lubricious material may be dried, for example, in air, in a heated environment, or in a vacuum oven. In some cases, ultraviolet radiation (UV), gamma radiation or e-beam irradiation may be used to aid in curing or cross-linking the lubricious material.

The lubricious material may be applied to the outside of a folded or unfolded balloon, to an inner surface of an expandable sheath, or to the outside surface of the expandable sheath. The lubricious material may be positioned between an inner surface of the expandable sheath and an outer surface of the balloon. Alternatively or additionally, the lubricious material may be applied to an inner surface of the coated stent. Patterning or selective coating with the lubricious material provides control over the degree of lubricity of the balloon relative to the inner diameter of the coated stent.

A balloon is positioned within an expandable sheath, as seen at block 520. The balloon may include a plurality of pleats. One or more balloons may be positioned within the expandable sheath. The balloon is wrapped with the expandable sheath to limit the shear forces on the stent coatings when the coated stent is being deployed. The expandable elastic sheath also helps refold the balloon prior to retraction into a guide catheter during deployment.

The pleats may be attached to the expandable sheath, as seen at block 530. The pleats may be attached to the inner surface of the expandable sheath, for example, using glue, stitching, or tacking. The pleats may be attached to the sheath at one or more points, for example, along an apex or outward tip of each pleat.

The expandable sheath and balloon are placed inside a drug-polymer coated stent, as seen at block 540. A fixture may be used to position the expandable sheath and balloon with the desired amount of margin at each end of the coated stent.

The stent deployment assembly is compressed, as seen at block 550. The drug-polymer coated stent is pressed against an outside surface of the expandable sheath. The stent deployment assembly is compressed, for example, by rolling the coated stent down to the desired diameter in a roll-down machine. The stent deployment assembly may be compressed, for example, by releasing a coated stent onto the sheath after the coated stent has been elastically enlarged.

A catheter is coupled to the stent deployment assembly, as seen at block 560. In one example, finished coated stents are reduced in diameter and placed into the distal end of the catheter in a process that forms an interference fit, which secures the stent, expandable sheath and balloon onto the catheter. The catheter with the stent is typically placed in a catheter package and sterilized prior to shipping and storing. Sterilization of the stent using conventional means is completed before clinical use.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A system for treating a vascular condition, comprising:
a catheter; and
a stent deployment assembly coupled to the catheter; the stent deployment assembly comprising a coated stent including a stent framework and a drug-polymer coating on at least a portion of the stent framework, an expandable sheath positioned adjacent to an inner surface of the coated stent, and at least one balloon positioned within the expandable sheath, wherein the expandable sheath expands against the stent framework as the balloon is inflated to deploy the coated stent within a vessel.

2. The system of claim 1 wherein the stent framework comprises one of a polymeric base or a metallic base.

3. The system of claim 2 wherein the metallic base comprises a material selected from the group consisting of stainless steel, nitinol, tantalum, MP35N alloy, platinum, titanium, a suitable biocompatible alloy, a suitable biocompatible material, and a combination thereof.

4. The system of claim 1 wherein the balloon includes a plurality of pleats.

5. The system of claim 4 wherein an apex of each pleat is attached to the inner surface of the expandable sheath.

6. The system of claim 1 wherein the balloon is lubriciously coupled to the expandable sheath.

7. The system of claim 1 wherein the expandable sheath comprises a material selected from the group consisting of nylon, polyurethane, polyethylene terephthalate, polyethylene, polytetrafluoroethylene, expanded polytetrafluoroethylene, an elastane, a thermoplastic elastomer, a woven polymeric fabric, or an expandable polymeric sheet.

8. The system of claim 1 further comprising:
a lubricious coating on at least a portion of an outer surface of the balloon.

9. The system of claim 8 wherein the lubricious coating comprises a material selected from the group consisting of phosphorylcholine, a hydrophilic coating, and a lubricious film.

10. The system of claim 1 further comprising:
a lubricious coating on at least a portion of an inner surface or an outer surface of the expandable sheath.

11. The system of claim 1 further comprising:
a lubricious coating on at least an inner surface of the coated stent.

12. The system of claim 1 wherein the expandable sheath contracts when the balloon is deflated.

13. The system of claim 1 further comprising:
a cap coat disposed on the drug-polymer coating.

14. A method of deploying a drug-polymer coated stent in a vessel, the method comprising:
inflating pleated portions of a balloon;
expanding a sheath with the inflating pleated portions of the balloon; and
deploying the drug-polymer coated stent with the expanding sheath.

15. The method of claim 14 wherein the sheath is lubriciously coupled to the pleated portions of the balloon.

16. The method of claim 14 wherein the sheath is attached to the pleated portions of the balloon.

17. The method of claim 14 further comprising:
deflating the pleated portions of the balloon after the coated stent is deployed; and
contracting the sheath while deflating the pleated portions.

18. A method of manufacturing a stent deployment assembly, the method comprising:
positioning a balloon within an expandable sheath, the balloon including a plurality of pleats; and
placing the expandable sheath and the balloon inside a drug-polymer coated stent; and
compressing the stent deployment assembly, wherein the drug-polymer coated stent is pressed against an outside surface of the expandable sheath.

19. The method of claim 18 further comprising:
attaching the plurality of pleats to an inner surface of the expandable sheath.

20. The method of claim 18 further comprising:
positioning a lubricious material between an inner surface of the expandable sheath and an outer surface of the balloon.

21. The method of claim 20 wherein positioning the lubricious material comprises coating at least a portion of the outer surface of the balloon.

22. The method of claim 18 further comprising:
positioning a lubricious material between an inner surface of the drug-polymer coated stent and an outer surface of the expandable sheath.

23. The method of claim 22 wherein positioning the lubricious material comprises coating at least a portion of the outer surface of the expandable sheath.

24. The method of claim 18 further comprising:
coupling a catheter to the stent deployment assembly.
